(19) United States
(12) Patent Application Publication
(10) Pub. No.: US 2006/0079781 A1
(43) Pub. Date: Apr. 13, 2006

(54) Ultrasonic apparatus for estimating artery parameters

(75) Inventors: Laurence Germond-Rouet, Paris (FR); Odile Bonnefous, Rueil-Malmaison (FR)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., Eindhoven (NL)

(21) Appl. No.: 10/538,621

(22) PCT Filed: Dec. 12, 2003
(86) PCT No.: PCT/IB03/05914

§ 371 Date: Jun. 10, 2005

(30) Foreign Application Priority Data
Dec. 18, 2002 (EP) 02293133.1

(51) Int. Cl. A61B 8/02

(52) U.S. Cl. 600/450

(57) ABSTRACT

The invention relates to an ultrasonic image processing system, for processing images in an image sequence representing a segment of artery explored along its longitudinal axis, said artery segment showing moving walls; this system comprising: acquisition means (21) for acquiring an ultrasonic image sequence of a segment of artery explored along its longitudinal axis and having walls moving in relation with the cardiac cycle; semi-automatic detection means (22) for detecting the artery walls in an image of the sequence; automatic rigid tracking means (23) for tracking the corresponding artery walls in other images of the sequence; evaluation means (24) for evaluating the artery wall motion and distensibility; and viewing means (154) for visualizing images. The invention further relates to an ultrasound examination apparatus having a curved array of transducer elements and coupled to this system, having viewing means to visualize the images.

### FIELD OF THE INVENTION

The invention relates to an ultrasonic imaging system and an ultrasonic examination apparatus having processing means for constructing and displaying an ultrasonic examination image sequence of an artery segment with indications of arterial parameters in function of the cardiac cycle The invention also relates to an image processing method having steps for operating this system and this apparatus. The invention is used in the field of ultrasonic imaging, to provide a cardio-vascular non-invasive medical tool for examining patients suspected to present anomalies of arteries and notably anomalies of the aorta such as aortic aneurysms.

### BACKGROUND OF THE INVENTION

An ultrasonic image processing method for calculating dilation curves related to an artery segment is already known from the patent U.S. Pat. No. 5,579,771 (Bonnefous, Dec. 3, 1996). This document describes a method for characterizing an artery segment by ultrasonic imaging, using an array of ultrasonic transducers that produces a sectional frame, which is formed by image lines of a number of successive parallel excitation lines extending perpendicularly to the artery axis. Said array is coupled to a transmitter/receiver circuit, which provides high frequency signals to a signal processing system. Said system determines the arterial walls radial velocity and displacement amplitude values and further determines an arterial dilation curve in function of location and time. Such a curve is constructed by points representing the arterial dilation value in the arterial radial direction Z, at a given location corresponding to an excitation line along the longitudinal X-axis of the artery, in function of excitation instants t, during a cardiac cycle. So, FIG. 4C of this document shows, superposed, the different dilation curves related to all the excitation lines of an ultrasonic signal corresponding to the examined artery segment, said lines being at regularly spaced locations along the X-axis of the artery.

The dilation curves are certainly very useful for the study of stenoses. A problem is that, in fact, for the study of aneurysms, the evaluation of distensibility is more exploitable by a cardiologist. The severity of an aneurysm may be estimated by considering its maximal diameter. Thus, the dilation information is useful. However, at the present time, cardiologists think that the mechanical stress acting on the artery walls at the location of the aneurysm is a very appropriate consideration. Thus, the distensibily information is a very appropriate consideration and is preferably used together with the dilation information.

Another problem is that the cited document relates to an image processing method based on image acquisition with ultrasound scanning lines that are perpendicular to the artery axis. This corresponds to the use of an ultrasound system for acquiring the ultrasound data with a linear array of transducer elements. This kind of system is appropriate for studying a shallow artery and a small segment of artery such as the carotid. This kind of system is not appropriate for the study of a deep and thick artery such as the aorta and particularly for the study of Abdominal Aortic Aneurysms (AAA). For studying the aorta and AAA, a curved array of transducer elements is preferably used. When the ultrasound data are acquired with a curved array, then the method disclosed in the cited document for calculating artery dilations cannot be directly used, since the scanning lines are no longer perpendicular to the artery axis.

Another problem is that the cited document only permits of disposing of constant values corresponding to the reference coordinates of the artery walls at the instants of zero dilation. This corresponds to a representation of the artery wall by a straight reference line at the instants of zero dilation. Such a linear reference representation is difficult to understand for the clinician. Hence, there is a need for a precise location and representation of the artery walls at these instants of zero dilation.

Abdominal Aortic Aneurysm (AAA) is defined by a doubling of the normal infra-renal aortic diameter. In order to early diagnosing aneurysms in aorta, the medical field has a need for non-invasive means for providing aorta images together with clear quantified indications of the aortic distensibility, which is a measure that is used by clinicians together with dilation information.

### SUMMARY OF THE INVENTION

In order to address the problem of finding new diagnosis information for the follow up of patients suspected to present Abdominal Aortic Aneurysms (AAAs), it is an object of the invention to propose an image processing system for the evaluation of parameters related to the tension and strain of the aneurysm walls. The present invention-proposes a system developed for AAAs that is specifically designed to provide clinicians with information on the motion of the aorta artery walls.

This image processing system is claimed in claim 1. This image processing system offers the advantage that the aorta wall behavior is made clearly visible together with the parameters that are useful for the clinician in the study of these Abdominal Aortic Aneurysms. This system has display means to visualize the images and constitutes a tool for non-invasive diagnostic of arterial wall anomalies. An ultrasonic diagnostic method having processing steps for operating this system and an ultrasound apparatus coupled to this system are claimed in dependent Claims.

### BRIEF DESCRIPTION OF THE FIGURES

Specific embodiments of the invention will be described in detail hereinafter with reference to the accompanying diagrammatic drawings; therein:

FIG. 1A shows a schematic representation of an aorta and Abdominal Aortic Aneurysm (AAA); FIG. 1B shows wall stress distribution on an Abdominal Aortic Aneurism;

FIG. 2 is a block diagram showing the main steps of the method of the invention;

FIG. 3 image in the image sequence, with wall borders drawn in ROIP and ROID;

FIG. 4 is a block diagram illustrating user interaction for drawing the artery wall borders;

FIG. 5 is an image of pixel costs for optimal path detection;

FIG. 6 illustrates the tracking propagation scheme;

FIG. 7 illustrates wall border tracking in the images of the sequence;

FIG. 8 is a block diagram of sub-steps of the tracking stage for finding wall borders using ROIP and ROID in the image sequence;

FIG. 9 is a block diagram of sub-steps of forward or backward rigid tracking in the sequence images;

FIGS. 10A and 10B are two views of the (2-D+t) potential function for ROIP and ROID;

FIG. 11 is an ultrasound image with indications of interactive selection of a diameter of an aorta for distensibility calculation;

FIG. 12 is an ultrasound image with indications of the dilations of an aorta;

FIG. 13 illustrates a box of information giving parameters related to a segment of aorta;

FIG. 14 is a block diagram of an examination apparatus with a viewing system having processing and display means for carrying out the method of the invention.

### DETAILED DESCRIPTION OF EMBODIMENTS

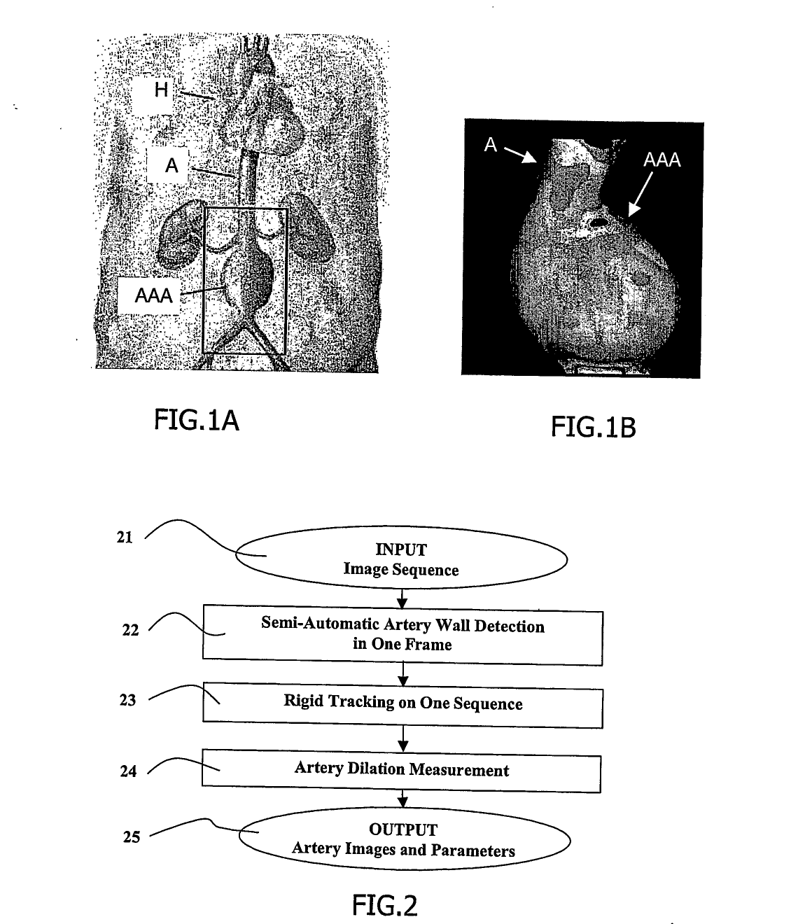

Referring to FIG. 1A, Abdominal Aortic Aneurysm AAA is defined by a doubling of the normal diameter of the infra-renal aorta A. The heart is denoted by H. The AAA abnormality is present in 5% of men aged over 65 years. Rupture of the aneurysm, the most common complication of AAA, is responsible for about 2% of deaths in men in this age group and is the tenth leading cause of death in men in Europe. Since most AAAs are asymptomatic until rupture occurs, up to 50% of all AAAs repairs are performed as an emergency operation. As the operative mortality for ruptured AAA is around 50%, and only a small fraction of patients with ruptured AAAs survive to reach hospital, the overall community mortality for ruptured AAAs is estimated at over 90%. For this reason, there is an increasing interest in the clinical and cost effectiveness of mass screening programs for AAAs. Acquired abdominal aortic aneurysms classically are characterized anatomically by an unparallelism of the aorta edges, resulting in an expanded and beating abdominal mass. The physiopathology consists of a loss of vascular contention, including a risk of rupture. Indeed, the aorta fulfills several haemodynamic functions of blood tissue distribution, damping of the pulse wave, etc. The most elementary of these functions is containing high pressure blood within the arterial lumen. Arterial wall aneurysmal diseases are characterized by partial loss of integrity called dilation or total loss of integrity corresponding to a rupture. Therefore, in order to early diagnosing aneurysms in aorta, the medical field has a need for non-invasive means for providing aorta images together with clear quantified indications of the aortic distensibility. Besides, it is important to use non-invasive means instead of invasive means because invasive means modifies the aorta pressure, hence the actual aorta distensibility.

The severity of Abdominal Aortic Aneurysm (AAAs) is generally clinically estimated by considering its maximal diameter. However, referring to FIG. 1B, which shows a wall stress distribution in different shades of colors on AAA shape, cardiologists tend to think that the mechanical stress acting on the artery walls at the location of the aneurysm is certainly a more appropriate consideration. Thus, distensibily information is a more appropriate consideration than dilation information. In fact, it is known that failure of any material occurs when the wall stress exceeds the strength of the material. Whereas operative indications for elective AAA repair are generally based on aneurysm size greater than 4.5 to 5 cm in diameter, the most frequently used medical approach is watchful waiting, whereby aneurysm diameter is periodically re-measured to detect expansion to a size warranting surgery of the patient. Now, it is also known that AAAs with a diameter less than 5 cm can rupture. Hence, there is a clear need for additional diagnosis information. Therefore, distensiblity information will be preferably used.

The present invention proposes an image processing system and an image processing method to provide aorta parameters for the evaluation of the tension and strain of the aneurysms walls. The system and the method are developed for AAAs and are specifically designed to provide clinicians with information on the behavior of the aortic artery walls.

The method is first described. This method permits of evaluating automatically, or with limited user interaction, and at any time in the image sequence, the position of the artery walls, in order to estimate the artery dilations and distensibility.

Referring to FIG. 2, the processing of an image sequence is divided into main steps of:
1) Acquisition of the image sequence 21;
2) Semi-automatic detection 22 of the aorta proximal and distal walls through user interaction combined with live-wire detection. This step is applied to one frame of the sequence;
3) Automatic rigid tracking 23 of the two walls in the sequence;
4) Evaluation 24 of the artery wall motions and dilations.
5) Display 25 of the results in the sequence.

This Abdominal Aortic Aneurysm Wall Motion (AAAWM) tool particularly comprises:

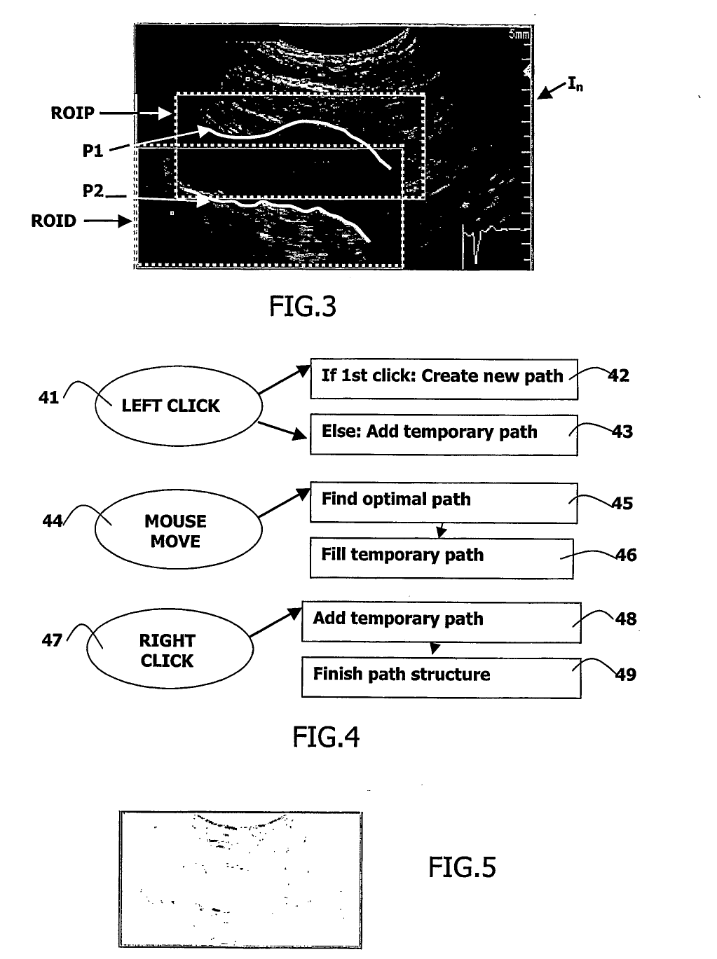

1) Acquisition 21 of a sequence of ultrasound images of a segment of artery, for instance a segment of aorta, using a linear curved array. Said artery segment has a longitudinal axis and is represented in grayscale images as illustrated by FIG. 3 or FIG. 11 or FIG. 12.

Referring to FIG. 14, an ultrasonic imaging system constructed in accordance to the principles of the present invention is shown in a block diagram form. In the example of embodiment that is described hereafter, this ultrasonic imaging system is used as a tool for the examination of the aorta. This ultrasonic imaging system comprises sub-systems to perform the image processing method of the invention for visualizing the arterial segment whose walls have radial movements and for quantifying its radial arterial dilation, which occurs under the influence of the blood pressure, at given locations of said arterial segment and in function of the different time instants during a cardiac cycle.

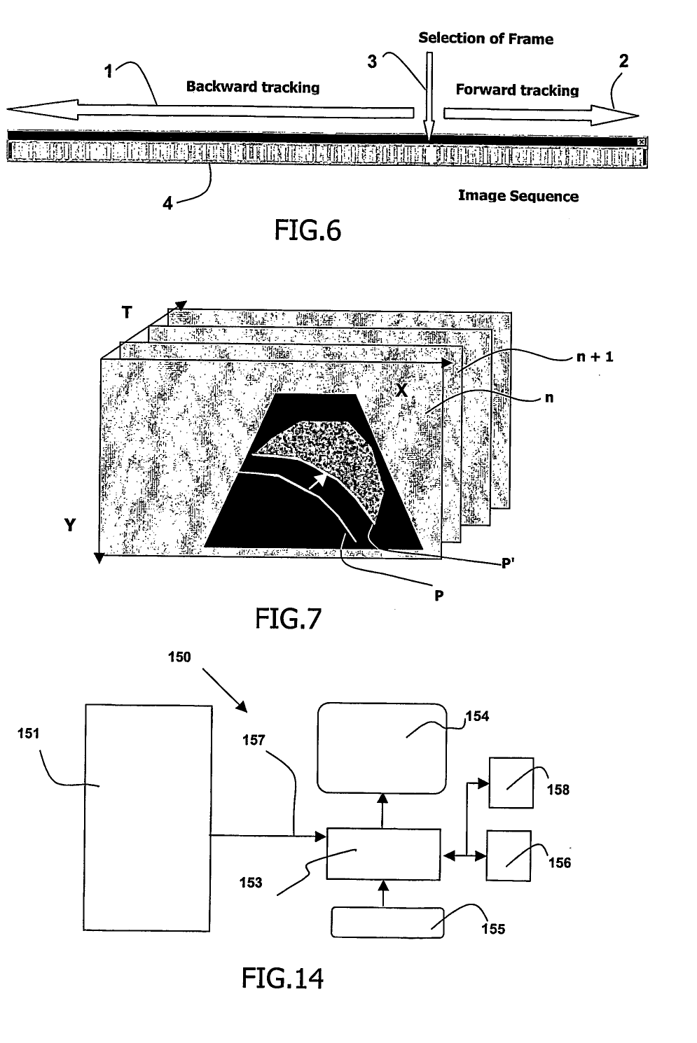

2) Semi-automatic segmentation 22 of the artery walls, based on the echo information, in one image of the sequence. As illustrated by the block diagram of FIG. 4, the user interacts in one image of the sequence and performs an assisted drawing of the artery boundaries using a technique called live-wire technique. The user can draw the artery wall borders in any image of the sequence as illustrated by FIG. 3. Thus, as illustrated by the tracking propagation scheme of FIG. 6, the user can choose as starting image, numbered n, where the definition of the boundaries appears with the best contrast. The live-wire technique includes the estimation of values called maxGrad and minGrad that represent respectively the maximum and minimum amplitude of the gradient in the image. Since the echo image is noisy, the image is first smoothed, using a gaussian filter, before gradient estimation. Then a cost function for the live-wire technique, adapted for the delineation of the aorta boundaries, is defined.

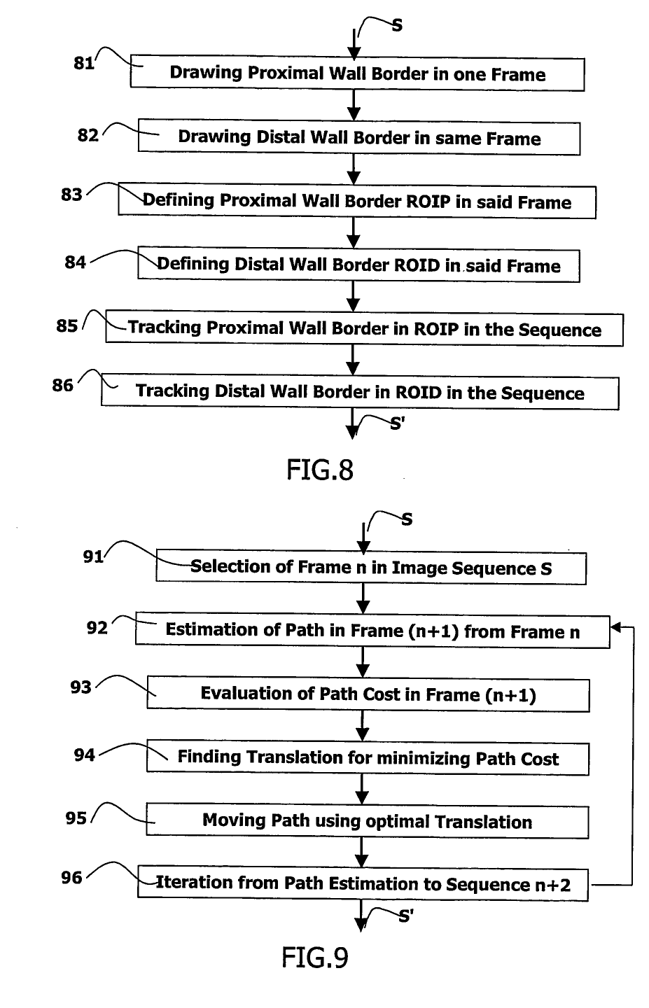

3) Automatic rigid tracking 23 of the artery wall position in the rest of the sequence, as illustrated by FIG. 7 and by the block diagrams of FIG. 8 and FIG. 9. The tracking is started at the starting image where the user has drawn the artery wall boundaries. Referring to the tracking propagation scheme of FIG. 6, if this starting image numbered n is in the middle of the image sequence 4, a forward tracking 2 and a backward tracking 1 are performed in order to provide a complete tracking for the whole image sequence 4. Regarding the forward tracking 2, the principle is to initialize the position of a structure in the current frame (n+1) at the same position as found in the previous frame n and then to move the structure in order to fit the boundaries of the current frame, as illustrated for example in FIG. 7. The movements of the structure are limited to vertical and horizontal translations. In order to determine the best fit in the current frame, an optimization criterion is used, based on the minimization of a cost function.

4) Additional processing 24 in order to measure the dilation and the distensibility of abdominal aorta with the ultrasound system using a linear curved array.

5) Output 25 of the parameters related to the artery under study as illustrated for example by the box of FIG. 11. The method of the invention can be used to measure the dilation of Abdominal Aortic Aneurysms (AAA) in the context of a surveillance of the growth, before treatment and after treatment with an endoprothesis. Local motion gradients show local strains. Low pulsatility in aneurysms is an indicator of non elastic arteries due to dilated walls and can be an indication of risk of rupture, which is a major health hazard. High pulsatility after stenting show a reperfusion of the aneurysm indicating a leak in the stent and necessitating further clinical intervention.

FIG. 14 shows a diagram of a medical viewing system 150 according to the invention for carrying out the steps of the image processing method described hereafter. The system has means 151 for acquiring digital image data of a sequence of images, and is coupled to computer means 153 for processing these data according to this image processing method. The data processing device 153 is programmed to implement a method of processing medical image data according to invention. In particular, the data processing device 153 has computing means and memory means to perform the steps of the method. A computer program product having pre-programmed instructions to carry out the method may also be implemented. Steps of the present method can be applied on stored medical images, for example for estimating medical parameters. The medical viewing system provides the image data by connection 157 to the system 153. The system provides processed image data to display means and/or storage means. The display means 154 may be a screen. The storage means may be a memory of the system 153. Said storage means may be alternately external storage means. This image viewing system 153 may comprise a suitably programmed computer, or a special purpose processor having circuit means such as LUTs, Memories, Filters, Logic Operators, that are arranged to perform the functions of the method steps according to the invention. The system 153 may also comprise a keyboard 155 and a mouse 156. Icones may be provided on the screen to be activated by mouse-clicks, or special pushbuttons may be provided on the system, to constitute control means 158 for the user to actuate the processing means of the system at chosen stages of the method. This medical viewing system 150 may be incorporated in an ultrasound examination apparatus 151. This medical examination apparatus 151 may include a bed on which the patient lies or another element for localizing the patient relative to the apparatus. The image data produced by the ultrasound examination apparatus 151 is fed to the medical viewing system 150.

The technical implementation of the above described steps for forming the Abdominal Aortic Aneurysm Wall Motion (AAAWM) tool is described hereafter more precisely. The terms artery wall border and “structure” have the same meaning and represent a segmented object.

As disclosed by the prior art cited in the introduction part, it is already known to determine artery dilations using a linear probe applied to the carotid artery of a patient. The known method is no more appropriate for carrying out the present method, since the probe used for the examination of the aorta of a patient is curved. The aortic aneurysms have very varying shapes and sizes depending on the subject under examination. As a consequence, the detection of the walls (structures) in the images of a sequence requires a very adaptive segmentation tool. In order to deal with the variability of the images, it is preferable to combine user interaction with a more automatic processing for the segmentation of an image. Thus, the user is asked to delineate the boundaries of the AAA walls in one image of the sequence that he can select. This delineation is semi-automatic and is based on a technique called “Live-Wire”, which is described in a publication entitled “User-Steered Image Segmentation Paradigms: Live Wire and Live Lane” by A. X. Falcao, J. K. Udupa, S. Samarasekera, S. Sharma, and B. E. Hirsh, in “Graphical Models and Image Processing 60, pp. 233-260, 1998”. The principle of the method of the present invention is to provide the automatic detection of a boundary located between successive points selected by the user on this boundary. The boundary detection is based on the optimization of a cost function.

Implementation of Step 1: Acquisition of an Image Sequence.

As a matter of example, the processed sequence of abdominal aortic aneurysms (AAA) has been acquired with a Tissue Doppler Imaging (TDI) modality, using a C5-2 probe and a Philips HDI5000 scanner.

Implementation of Step 2: Semi-Automatic Edge Detection in One Selected Frame.

Referring to FIG. 14, and to FIG. 3, for assisted-drawing of a boundary, the user can click on the left or right button of the mouse 156 and move the mouse, while visualizing the starting image selected in the sequence of images numbered n that is displayed on the screen 154. Referring to FIG. 4, the left clicks 41 are used to begin a boundary and to select intermediate points in a boundary. The right clicks 47 are used to terminate a boundary. The boundaries are stored in “path” structures. The handling of the different user interactions is described bellow:

With the left click 41, if it is the first click: creation 42 of a new path structure; else: addition 43 of the temporary path to the path structure.

With the mouse move 44: Finding 45 the optimal path between the last left click and the current cursor position; or filling 46 a temporary path with the result.

With the right click 47: Addition 48 of the temporary path to the path structure; or finishing 49 the path.

A cost function is used to determine the optimal path between two successive positions of the mouse. The first position is always associated to a click of the user. The second position can either be the current position of the mouse or a click of the user. This allows to showing to the user in real-time where the optimal path is found by the path search technique. The cost of a path between two positions of the mouse is the sum of the costs of the individual pixels that constitute the path. Since the goal of the path search technique is to minimize the cost of a path, the costs of the individual pixels at boundary positions must be small. The individual costs are based on the gradient of the echo image. Since the echo image is rather noisy, it is first smoothed, using a gaussian filter, before the gradient estimation. The cost of a pixel is defined by the following formula:

$$cos t ( {pixel}_{i} ) = 255 * \frac{max Grad - grad ( {pixel}_{i} )}{max Grad - min Grad} \qquad ( 1 )$$

where maxGrad and minGrad represent respectively the maximum and minimum amplitude of the gradient. The cost of individual pixels is calculated for each pixel of the image where the user interacts. FIG. 5 shows an image of pixel costs for optimal path detection, where low costs are in dark and represent image boundaries.

Implementation of Step 3: Structure Rigid Tracking in the Image Sequence S.

In fact, the aortic aneurysms do not considerably deform through an image sequence. As a first approximation, a rigid tracking of the motion, limited to translations, can be used to automatically detect the structures in the remainder of the sequence. The tracking is initialized with the result of the semi-automatic segmentation provided by the user in the initially selected frame of the sequence. The proximal and the distal walls, also called structures, are individually tracked in the whole sequence.

Referring to FIG. 8, the rigid tracking comprises general sub-steps among which:
Sub-steps 81 and 82 of drawing proximal and distal wall borders, called structures, in the selected Frame n, called starting Frame;

Sub-steps 83, 84 of defining regions of interest, denoted by ROI, around each structure: FIG. 3 illustrates the definition of one ROI called ROIP for the determination of the proximal wall border denoted by P1, and the definition of one ROI called ROID for the determination of the distal wall border denoted by P2. Same ROIs are used in all the frames of the sequence;
Sub-steps 85, 86 of tracking the proximal and distal wall borders in ROIP and ROID respectively.

Referring to FIG. 7 and FIG. 9, the rigid tracking is initialized in sub-step 91 with the structures semi-automatically segmented in the selected frame denoted by n of the sequence S. The tracking starts from the selected frame n and is propagated towards the beginning of the sequence according to a direction 1 called backward tracking, as well as towards the end of the sequence according to a direction 2 called forward tracking as illustrated by the scheme of FIG. 6. The iteration of the rigid tracking for one structure in a sequence is described with reference to FIG. 6 and FIG. 9. This description is limited to the forward tracking 2. The technique for backward tracking 1 is fully symmetric. The rigid tracking comprises detailed sub-steps of:

In a sequence S, selection 91 of a starting Frame n in the image sequence 4 and drawing a path as previously described in reference to Step 2 illustrated by FIG. 4 and detailed sub-steps 41 to 49;

Referring to FIG. 7, using the position P of the path in Frame n, as an initial estimate of the position of the path with the coordinates X, Y, performing an estimation 92 of the path P′ in the next frame (n+1); the principle is to initialize, in sub-step 92, the position of a structure in the current frame (n+1) at the same position as found in the previous frame n and then to move the structure in order to fit the boundaries of the current frame;

Evaluation 93 of the cost of the path at the current position in frame (n+1) as the sum of the potentials of each point of the path; in order to determine the best fit in the current frame, an optimization criterion is defined. This criterion is the minimization of a cost function in sub-step 93. Similarly to the principle used in previous step 2, the cost of a structure is defined as the sum of the costs of all the pixels of the structure;

Finding 94 the translation, among a limited number of possible translations, that minimizes the cost of the path; the search for the optimal translation is implemented with a full exploration of the possible translations within the limits of allowed translations in sub-step 94; the movements of the structure are limited to vertical and horizontal translations;

Moving 95 the path by the optimal translation found at the previous step; the hypothesis of motion continuity is used to reduce the area where the translations are considered in sub-step 95;

Iteration 96 from the sub-step 92 of path estimation until the end of the sequence.

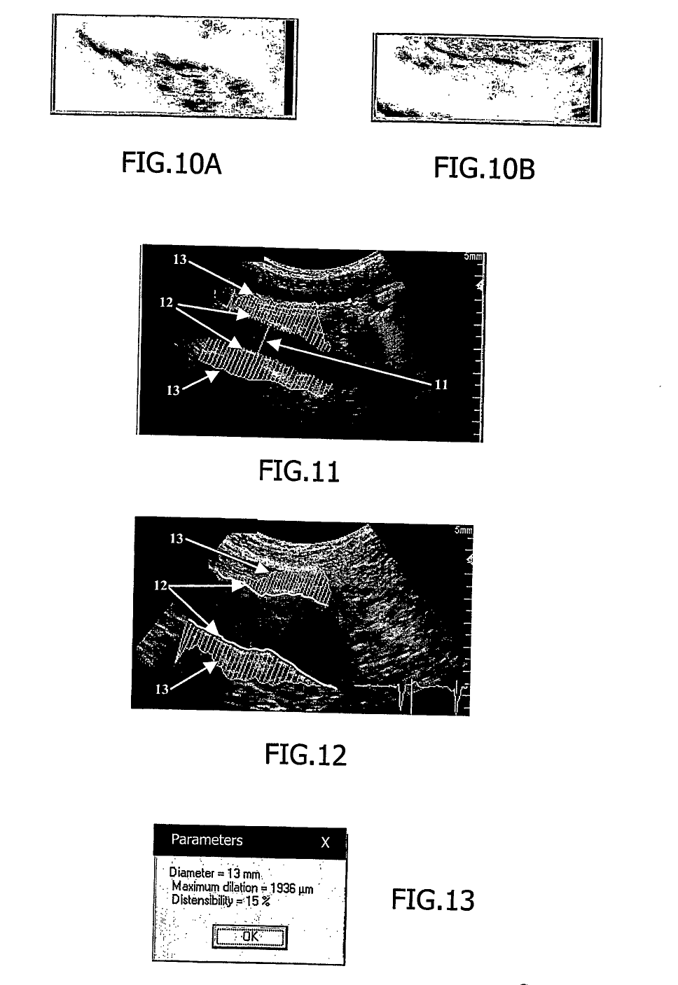

The cost function used for the spatio-temporal tracking of the structures in the sequence is based on individual pixel costs calculated as in equation (1). The main difference is that the gradient is calculated for all the frames of the sequence and that these frames are considered as a two-dimensional (X,Y)+time (t) volume [(2-D+t) volume] and not as individual frames. This provides a spatio-temporal estimation of the gradient in the sequence. This technique is interesting because it smoothes the gradient in time direction, which ensures more motion continuity between successive frames. Since the computation of the (2-D+t) gradient is the most time consuming step of the whole processing of the AAAWM tool, this computation is performed in the regions of interest denoted by ROIP for the proximal wall border determination, and ROID for the distal wall border determination. Same ROIs are used in all the frames of the sequence and thus defines the (2-D+t) image. Cost images for ROIP and ROID are represented respectively in FIGS. 10A and 10B, which show 2-D views of the (2-D+t) potential function for each ROI.

Implementation of Step 4: Evaluation of the Artery Dilations.

The ultrasound color information used to process the wall motion is the ultrasound raw color data. It is composed of the lines of the ultrasound color scanning and, for each line, the estimates of velocities in depth. The distensibility is interactively measured by selecting two opposite points on the arterial walls in an image. The two points are linked by segment 11, illustrated as shown in FIG. 11, to represent the diameter at the selected position. A pre-requisite for the evaluation of the distensibility d, is that the dilations of the artery walls have been computed, as illustrated by FIG. 11 and FIG. 12.

The dilation estimation is the result of the difference of motion between two structures for each ultrasound color line. The dilations are calculated, as disclosed in the document cited as prior art, in order to provide input data for the interface of the application, as illustrated by the image of FIG. 12 and the box of FIG. 13. The distensibility d is computed at the selected diameter position using the following formula:

$$d = \frac{dilation}{diameter} * 100 \% \qquad ( 2 )$$

Implementation of Step 5: Display of the Images and Parameters.

In order to represent the motion in the images, a choice must be made regarding the estimated direction of the motion. In this application, the hypothesis is that the motion of the artery walls is perpendicular to the artery principal axis. The display provided in each frame of the sequence is limited to two types of information. The first type is the structure location. The proximal and distal walls, called structures 12, are represented in colors, preferably in the same color, called first color. Then, the motion of each wall along each ultrasound color line is preferably represented in another color, called second color. The reference line for a null motion is the structure itself and the amplitudes are represented starting from the structure position. The representation of the lines of the second color allows to understanding the direction of projection that was selected for each motion amplitude. The lines of the second color are interconnected to represent the overall shape 13 of the motion between ultrasound color lines. After the processing, the results are summarized on a dedicated interface, such as in FIG. 12 and FIG. 13. FIG. 12 represents an echo image in gray level corresponding to the user-selected frame, combined with the segmentation result and the dilation amplitudes. The image of FIG. 12 also represents dilation amplitudes. The box of FIG. 13 displays the artery parameters.

1. Ultrasonic image processing system, for processing images in an image sequence representing a segment of artery explored along its longitudinal axis, said artery segment showing moving walls; this system comprising:
semi-automatic detection means for detecting the artery walls in an image of the sequence;
automatic rigid tracking means for tracking the corresponding artery walls in other images of the sequence;
evaluation means for evaluating the artery wall motion and distensibility; and
viewing means for visualizing images.

2. The system of claim 1, wherein the semi-automatic detection means is a user assisted artery wall detection means comprising user interaction means for:
selecting a reference image as starting image among the images of the sequence;
drawing lines, called paths, representing the artery walls in the starting image, assisted by a path search technique based on the minimization of a cost function.

3. The system of claim 2, wherein the user interaction means for drawing a path representing a wall comprises means for:
selecting a starting pixel in the starting image for creating a new path structure;
drawing a portion of path between the starting pixel and a second selected pixel in the starting image;
evaluating the cost function of the portion of path as the sum of the cost of the individual pixels that constitute the path;
selecting the optimal path as the path that minimizes the cost function;
memorizing the optimal path;
drawing portions of path between successive pixels; and performing the operations of evaluating the respective cost functions, selecting the optimal paths and memorizing the optimal paths until a complete path is drawn for the artery wall.

4. The system of claim 3, having means for estimating the cost of the individual pixels based on the gradient at the pixel in the ultrasonic image.

5. The system of one claim 3, wherein the automatic rigid tracking means for tracking the corresponding artery walls in other images of the sequence comprises means of path finding including means for:
defining regions of interest (ROIP, ROID) around the paths drawn in the starting image and using the same regions of interest in the other images of the sequence;
selecting a current image next to the starting image;
initializing the tracking of the paths in the current image by using the positions of the paths in the starting image;
applying translations to said initial paths in the current image to fit the walls in the current image;
evaluating the cost of the paths in the current image using the same cost function as in the starting image and finding the translations that minimizes the cost function;
iterating these path finding steps until the beginning and the end of the sequence are reached.

6. The system of claim 5, wherein means for evaluating the cost performs cost evaluation of the individual pixels based on the gradient at the pixel in the ultrasonic images calculated for all the images of the sequence, considered as a two-dimensional plus time volume.

7. The system of one of claim 1, comprising computation means for calculating the dilation of the artery along the ultrasound beams in the images of the sequence using the segmentation of the walls performed by path finding with semi-automatic detection and rigid tracking.

8. The system of claim 7, comprising computation means for calculating the distensibility as the ratio of the dilation by the diameter of the artery.

9. A system as claimed in claim 1, having color display means to display colored paths for the artery walls and colored patterns for the wall dilation, superimposed on the ultrasonic images.

10. A system as claimed in claim 1, comprising a suitably programmed computer of a workstation or a special purpose processor having circuit means, which are arranged to process ultrasonic images, having means to display the processed images, and having a user interface such as a mouse or a keyboard to permit the user of interacting on the respective images of the sequence in order to display the quantified parameters related to the artery walls.

11. A computer program product comprising a set of instructions to be used in a system as claimed claim 1.

12. A system as claimed in claim 1, wherein the transducer array is a curved transducer array.

13. An ultrasonic medical image processing method comprising steps of acquiring a sequence of ultrasound images, using an array of transducer elements, and steps for detecting anomalies in arteries comprising:
semi-automatic detection of the artery walls in a reference image of the sequence;
automatic rigid tracking of the corresponding artery walls in other images of the sequence; evaluation of the artery wall motion and distensibility; and
visualization of images.

14. An ultrasound examination apparatus having means to acquired ultrasound images and coupled to an image processing system according to claim 1.
